(19) United States
(12) Patent Application Publication
(10) Pub. No.: US 2020/0308087 A1
(43) Pub. Date: Oct. 1, 2020

(54) Processes for Producing Xylenes

(71) Applicant: ExxonMobil Chemical Patents Inc., Baytown, TX (US)

(72) Inventors: Daniel J. Benedict, Houston, TX (US); Jeffrey L. Andrews, Houston, TX (US); Mika L. Shiramizu, Houston, TX (US); Nora B. Sennett, Ann Arbor, MI (US)

(21) Appl. No.: 16/817,756

(22) Filed: Mar. 13, 2020

Related U.S. Application Data
(60) Provisional application No. 62/824,470, filed on Mar. 27, 2019.

(51) Int. Cl. C07C 5/27

(52) U.S. Cl. CPC C07C 5/2791; C07C 15/08; C07C 2529/40

(57) ABSTRACT

A process for making p-xylene comprising co-feeding toluene with C8 aromatics into a vapor phase isomerization unit can improve xylenes loss and benzene selectivity in ethylbenzene conversion

### CROSS-REFERENCE TO RELATED APPLICATIONS

This application claims priority to U.S. Ser. No. 62/824,470, filed Mar. 27, 2019, herein incorporated by reference.

### FIELD

This disclosure relates to processes for producing xylenes. In particular, this disclosure relates to processes for vapor phase isomerization of C8 aromatic hydrocarbons which include ethylbenzene conversion for producing xylenes, particularly p-xylene.

### BACKGROUND

Para-xylene (“PX” or p-xylene) is a valuable chemical feedstock and is used mainly for the production of terephthalic acid and polyethylene terephthalate resins, in order to provide synthetic textiles, bottles, and plastic materials among other industrial applications. p-Xylene may be derived from mixtures of C₈ aromatics separated from such raw materials as petroleum naphtha, particularly reformates. The C₈ aromatic fractions from these sources vary quite widely in composition but, in the case of a reformate stream, can include 10 wt % to 32 wt % of ethylbenzene (“EB”) and xylenes (including about 50 wt % of meta-xylene (“MX” or m-xylene) and 25 wt % each of PX and ortho-xylene (“OX” or o-xylene). A major source of xylenes is a catalytic reformate, which is produced by contacting petroleum naphtha with a hydrogenation/dehydrogenation catalyst on a support. The resulting reformate is a complex mixture of paraffins and C₆ to C₈ aromatics, in addition to a significant quantity of heavier aromatic hydrocarbons. As commercial applications of p-xylene have increased, combining physical separation with chemical isomerization of the other xylene isomers to increase the yield of the desired para-isomer has become crucial to a greater extent. Although benzene and toluene are important aromatic hydrocarbons, the demand for xylenes, particularly p-xylene, currently is growing at an annual rate of about 5% to 7%, thus subjecting many efforts to develop efficient and cost-effective p-xylene formation and isolation processes.

However, with the boiling point of EB being near to the boiling points of p-xylene and m-xylene, a complete removal of EB from a C8+-aromatics containing fraction by conventional methods (e.g., distillation) is impractical because it would lead to a substantial amount of xylenes removed along with the EB exiting the distillation column. Thus, the xylenes-containing feed supplied to the isomerization unit is often allowed to contain EB at significant quantity. To prevent EB from accumulating in the C8 aromatics isomerization/recycle loop, dealkylation of EB into benzene and ethylene in the isomerization unit has become important.

In addition to dealkylation, the EB may be converted by isomerization or transalkylation. Whether an isomerization process will convert EB can depend upon the reactor conditions, including the type of process and catalyst selected. Accordingly, in many xylenes production processes, the conversion of EB is not maximized, mostly due to the need to control the competing reactions which convert xylenes to other compounds such as toluene and C₉+-aromatics.

Commercial xylenes production processes typically contain catalyst that may need constant upgrades, such as developing “selectivated” catalysts (e.g., catalysts with tailored/modified pore sizes in order to enhance their molecular-sieving or shape-selective capability), suitable for better diffusion of EB molecules for the dealkylation, isomerization, or other EB conversion processes. Despite such advances for these processes, xylenes loss (e.g., p-xylenes loss) remains considerable due to the fact that p-xylene is not sterically hindered enough to prevent its diffusion along with EB through the pores of the catalyst. After entering the catalyst pores, p-xylene can undergo side reactions such as transalkylation-type reactions resulting in p-xylenes loss and total xylenes loss. In addition, the xylenes loss can be particularly significant when a xylenes isomerization process feed contains high p-xylene content (such as in cases when a crystallizer is used for p-xylene recovery, or when an upstream p-xylene recovery molecular sieve nears its end-of-life).

There is a need for new and improved p-xylene production processes, especially isomerization processes, enabling higher EB conversion, while further reducing xylenes loss, and further maximizing the production of p-xylene, while minimizing the associated capital and operating costs of the p-xylene formation process.

References for citing in an Information Disclosure Statement (37 CFR 1.97(h)): W.O. 2000/010944; U.S. Pat. Nos. 5,689,027; 6,504,072; 6,689,929; U.S. Pub. No. 2002/0082462 A1; U.S. Pub. No. 2014/0350316 A1; U.S. Pub. No. 2015/0376086 A1; U.S. Pub. No. 2015/0376087 A1; U.S. Pub. No. 2016/0101405 A1; U.S. Pub. No. 2016/0185686 A1; U.S. Pub. No. 2016/0264495 A1; U.S. Pub. No. 2017/0210683 A1; U.S. Pat. No. 9,738,573; U.S. 9,708,233; U.S. Pat. Nos. 9,434,661; 9,321,029; 9,302,953; 9,295,970; 9,249,068; 9,227,891; 9,205,401; 9,156,749; 9,012,711; 7,915,471; 6,448,459; U.S. Pub. No. 2017/0204024 A1.

### SUMMARY

It has been found, in a surprising manner, that by co-feeding a quantity of toluene into a vapor phase isomerization reactor for isomerizing a mixture of xylenes and EB, the xylenes loss in the isomerization step can be significantly reduced, especially if the isomerization feed comprises p-xylene at a high concentration. Moreover, feeding toluene at a substantial quantity to a vapor phase isomerization reactor can have the added benefit of maintaining a desirable WHSV of the process. Still more surprisingly, co-feeding additional toluene resulted in higher selectivity for benzene in EB conversion in the vapor phase isomerization reactor.

Thus, a first aspect of this disclosure relates to a process for producing p-xylene, the process comprising: (I) supplying a first C8 aromatics mixture and toluene into a vapor-phase isomerization (“VPI”) unit, wherein: the first C8 aromatics mixture comprises ethylbenzene, p-xylene, and optionally toluene at a toluene concentration lower than 2 wt %, based on the total weight of the first C8 aromatics mixture; and the total weight of toluene fed into the VPI unit, including any toluene present in the first C8 aromatics mixture, is at least 2 wt % of the total weight of the C8 aromatics mixture and the toluene fed into the VPI unit; (II) operating the VPI unit under VPI conditions effective to isomerize the xylenes and dealkylate ethylbenzene in the presence of a first catalyst system in the VPI unit; and (III) obtaining a first product mixture effluent from the VPI unit, the first product mixture effluent being depleted in ethylbenzene relative to the first C8 aromatics mixture.

A second aspect of this disclosure relates to a process for producing p-xylene, the process comprising: (A) supplying a first C8 aromatics mixture into a vapor-phase isomerization (“VPI”) unit having a first catalyst system disposed therein, the first C8 aromatics mixture comprising ethylbenzene, p-xylene, and optionally toluene at a toluene concentration lower than 2 wt % based on the total weight of the first C8 aromatics mixture; (B) operating the VPI unit under conditions effective to isomerize at least a portion of the xylenes and dealkylate at least a portion of the ethylbenzene in the presence of the first catalyst system; (C) obtaining a first product mixture effluent from the VPI unit, the first product mixture effluent being depleted in ethylbenzene relative to the first C8 aromatics mixture; (D) determining a concentration of p-xylene of C(pX) in the first C8 aromatics mixture based on the total weight of C8 aromatics mixture; and (E) if C(pX)≥5 wt %, supplying additional toluene to the VPI unit such that the total weight of toluene fed into the VPI unit, including any toluene contained in the first aromatics mixture, is at least 2 wt % of the total weight of the C8 aromatics mixture and the toluene fed into the VPI unit.

### BRIEF DESCRIPTION OF THE DRAWINGS

FIG. 1 is a simplified schematic flow diagram of a process and system for producing p-xylene from catalytic reformate, according to one embodiment of this disclosure.

FIG. 2 is a graph showing xylenes loss (wt %) as a function of EB conversion (%) in two comparative examples and three examples of this disclosure.

FIG. 3 is a graph showing benzene concentration in the isomerization product mixture effluent (wt %) as a function of EB conversion (%) in the two comparative examples and three examples of this disclosure shown in FIG. 2.

FIG. 4 is a graph showing selectivity to benzene (mol %) in the EB conversion process as a function of the EB conversion (%) in the two comparative examples and three examples of this disclosure shown in FIGS. 2 and 3.

### DETAILED DESCRIPTION

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other step, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite article “a” or “an” shall mean “at least one” unless specified to the contrary or the context clearly indicates otherwise. Thus, embodiments using “an ether” include embodiments where one, two or more ethers are used, unless specified to the contrary or the context clearly indicates that only one ether is used.

For the purposes of this disclosure, the nomenclature of elements is pursuant to the version of Periodic Table of Elements as described in CHEMICAL AND ENGINEERING NEWS, 63(5), pg. 27 (1985).

The term “Cn” compound or group, wherein n is a positive integer, means a compound or a group comprising carbon atoms therein at the number of n. Thus, a “Cm to Cn” alkyl means an alkyl group comprising carbon atoms therein at a number in a range from m to n. Thus, a C1-C3 alkyl means methyl, ethyl, n-propyl, or 1-methylethyl-. The term “Cn+” compound or group, wherein n is a positive integer, means a compound or a group comprising carbon atoms therein at the number of equal to or greater than n. The term “Cn-” compound or group, wherein n is a positive integer, means a compound or a group comprising carbon atoms therein at the number of equal to or lower than n.

The term “conversion” refers to the degree to which a given reactant in a particular reaction (e.g., dealkylation, transalkylation, etc.) is converted to products. Thus 100% conversion of EB refers to complete consumption of EB, and 0% conversion of EB refers to no measurable reaction of EB.

The term “selectivity” refers to the degree to which a particular reaction forms a specific product, rather than another product. For example, for the conversion of EB, 50% EB selectivity to benzene means that 50% of the products formed are benzene. The selectivity is based on the product formed, regardless of the conversion of the particular reaction. The selectivity for a given product produced from a given reactant can be defined as mole percent (mol %) of that product relative to the total moles of the products formed from the given reactant in the reaction.

This disclosure provides an improved process and system for producing p-xylene including co-feeding a quantity of toluene with a p-xylene-depleted first C8 aromatics mixture into a vapor-phase isomerization (“VPI”) unit. Such co-feeding of toluene can unexpectedly reduce xylenes loss and enhance benzene selectivity in the VPI process. The process of this disclosure can be particularly advantageous when the first C8 aromatics mixture comprises p-xylene at an elevated concentration, e.g., ≥5 wt %, based on the total weight of the first C8 aromatics mixture. The process of this disclosure can be particularly advantageous where the VPI unit receives the first C8 aromatics mixture at a quantity substantially below design capacity. The process of this disclosure can be especially advantageous in a system including both a VPI unit and a liquid-phase isomerization (“LPI”) unit, where the LPI may divert a portion of the p-xylene-depleted C8 aromatics feed from the VPI unit, potentially resulting in the VPI unit running at below design capacity. This disclosure further provides an improved process and system for producing p-xylene comprising monitoring/determining the p-xylene concentration in the p-xylene-depleted first C8 aromatics mixture, and co-feeding toluene to the VPI unit if p-xylene concentration is no less than 5 wt % based on the total weight of the first C8 aromatics mixture.

### I. Xylenes Isomerization in General

High purity p-xylene products are typically produced by separating p-xylene from a p-xylene-rich aromatic hydrocarbon mixture comprising p-xylene, o-xylene, m-xylene, and optionally EB in a p-xylene separation/recovery system. The p-xylene recovery system can comprise, e.g., a crystallizer and/or an adsorption chromatography separating system known in the prior art. The p-xylene-depleted stream produced from the p-xylene recovery system (the “filtrate” from a crystallizer upon separation of the p-xylene crystals, or the “raffinate” from the adsorption chromatography separating system, collectively “raffinate” in this disclosure) is rich in m-xylene and o-xylene, and contains p-xylene at a concentration typically below its concentration in an equilibrium mixture consisting of m-xylene, o-xylene, and p-xylene. To increase yield of p-xylene, the raffinate stream may be fed into an isomerization unit, where the xylenes undergo isomerization reactions in contacting an isomerization catalyst system to produce an isomerized effluent rich in p-xylene compared to the raffinate. At least a portion of the isomerized effluent, after optional separation and removal of lighter hydrocarbons that may be produced in the isomerization unit, can be recycled to the p-xylene recovery system, forming a “xylenes loop.”

Xylenes isomerization can be carried out under conditions where the C8 aromatic hydrocarbons are substantially in vapor phase in the presence of an isomerization catalyst (vapor-phase isomerization, or “VPI”).

Exemplary VPI processes and catalyst systems are described in, e.g., U.S. Pat. Nos. 4,899,011 and 5,689,027, WO2006/022991, WO2007/127049, and WO2013/032630, the contents of all of which are incorporated herein by reference in their entirety.

Alternatively or additionally, newer generation technology have been developed to allow xylenes isomerization at significantly lower temperature in the presence of an isomerization catalyst, where the C8 aromatic hydrocarbons are substantially in liquid phase (liquid-phase isomerization, or “LPI”). The use of LPI vs. traditional VPI can reduce the number of phase changes (liquid to/from vapor) required to process the C8 aromatic feed. This provides the process with sustainability advantages in the form of significant energy savings. It would be highly advantageous for any p-xylene production plant to deploy a LPI unit, in addition to or in lieu of a VPI unit. For existing p-xylene production facilities lacking a LPI, it would be highly advantageous to add a LPI unit to compliment the VPI unit or replace the VPI unit, due the many advantages described above.

Exemplary LPI processes and catalyst systems useful therefor are described in U.S. Patent Application Publication Nos. 2011/0263918 and 2011/0319688, the contents of both of which are incorporated herein by reference in their entirety. Exemplary LPI isomerization conditions include a temperature of from about 220° C. to 480° C. (alternatively from about 220° C. to about 350° C., alternatively from about 350° C. to 480° C.), a pressure from about 446 kPa to about 3,500 kPa (alternatively from about 1,300 kPa to about 2,860 kPa), a WHSV of from about 0.5 hr-1 to about 50 hr-1 (such as from about 3 hr-1 to about 50 hr⁻¹) and a hydrogen to hydrocarbon molar ratio from about 0.7 to about 5. The WHSV is based on the weight of catalyst composition, i.e., the total weight of active catalyst and, if used, binder therefor.

### II. EB Conversion in Xylenes Isomerization and VPI in General

In the petrochemical processes for producing the xylenes-rich feed supplied to the p-xylene recovery system, such as a catalytic reforming process, EB may be produced at significant quantity along with the xylenes. As an isomer of the xylenes, EB has a boiling point very close to those of the xylenes. It would be impractical to completely separate EB from the xylenes by distillation. Thus, to prevent unnecessary loss of xylenes, the xylenes-rich feed supplied to the p-xylene recovery system derived from a catalytic reformer effluent are often allowed to contain EB at significant concentrations. As such, the raffinate from the p-xylene recovery system can comprise EB at significant concentrations.

It would be highly desirable to convert EB in the raffinate to higher value molecules such as xylenes, benzene, or toluene in the isomerization step. However, catalysts effective for isomerizing the xylene molecules between and among them may not be as effective in converting EB. This is especially true at a low temperature such as in a LPI process. At higher temperature process, such as in a VPI process, EB can be more readily converted into other molecules. EB can accumulate in the xylenes loop if not reduced or purged, which can be undesirable. Therefore, the inclusion of a VPI in a p-xylene production facility may be desired, especially if the feed subject to isomerization is rich in EB.

Without intending to be bound by a particular theory, it is believed that in a VPI process, EB conversion is carried out either by dealkylation to benzene or by isomerization to xylenes. Where the preferred mechanism is dealkylation to benzene, any conventional catalytic process for the dealkylation of EB can be used. However, in one preferred embodiment, the dealkylation is effected in the presence of a catalyst comprising an intermediate pore size zeolite (that is having a Constraint Index of 1 to 12 as defined in U.S. Pat. No. 4,016,218) and a hydrogenation component, optionally in combination with a non-acidic binder, such as silica. Examples of suitable intermediate pore size zeolites include ZSM-5 (U.S. Pat. No. 3,702,886 and Re. 29,948); ZSM-11 (U.S. Pat. No. 3,709,979); ZS M-12 (U.S. Pat. No. 3,832,449); ZSM-21 (U.S. Pat. No. 4,046,859); ZSM-22 (U.S. Pat. No. 4,556,477); ZSM-23 (U.S. Pat. No. 4,076,842); ZSM-35 (U.S. Pat. No. 4,016,245); ZSM-38 (U.S. Pat. No. 4,406,859); ZSM-48 (U.S. Pat. No. 4,397,827); ZSM-57 (U.S. Pat. No. 4,046,685); and ZSM-58 (U.S. Pat. No. 4,417,780). Examples of suitable hydrogenation components include the oxide, hydroxide, sulfide, or free metal (i.e., zero valency) of Group 8-10 metals (i.e., Pt, Pd, Ir, Rh, Os, Ru, Ni, Co, and Fe), Group 14 metals (i.e., Sn and Pb), Group 15 metals (i.e., Sb and Bi), and Group 7 metals (i.e., Mn, Tc, and Re), and combinations and mixtures thereof. Noble metals (i.e., Pt, Pd, Ir, and Rh) or Re are preferred hydrogenation components. Combinations of catalytic forms of such noble or non-noble metal, such as combinations of Pt with Sn, may be used.

In one preferred embodiment, the dealkylation catalyst is selectivated, either before introduction into the dealkylation reactor or in-situ in the reactor, by contacting the catalyst with a selectivating agent, such as at least one organosilicon in a liquid carrier and subsequently calcining the selectivated catalyst at a temperature of 350 to 550° C. The selectivation procedure alters the diffusion characteristics of the catalyst such that the catalyst can require at least 50 minutes to sorb 30% of its equilibrium capacity of o-xylene at 120° C. and at an o-xylene partial pressure of 4.5+/−0.8 mm of mercury. One example of a selectivated EB dealkylation catalyst is described in U.S. Pat. No. 5,516,956, incorporated herein by reference.

Exemplary conditions for the vapor phase dealkylation of EB using the above-described catalyst include a temperature from about 400° F. to about 1000° F. (204 to 538° C.), a pressure from about 0 to about 1,000 psig (100 to 7000 kPa), a weight hourly space velocity (WHSV) of between about 0.1 and about 200 hr⁻¹, and a hydrogen to hydrocarbon molar ratio from about 0.5 to about 10. Preferably, these conversion conditions include a temperature of from about 660° F. to about 900° F. (350° C. to 480° C.), a pressure from about 50 to about 400 psig (446 to 2860 kPa), a WHSV of between about 3 and about 50 hr⁻¹ and a hydrogen to hydrocarbon molar ratio from about 0.7 to about 5. The WHSV is based on the weight of catalyst composition, i.e., the total weight of active catalyst and, if used, binder therefor. The conversion conditions are selected so that the C₈ aromatic hydrocarbon-containing feed is substantially in the vapor phase in the VPI unit.

### III. The Process of This Disclosure in Relation to a VPI Process in a VPI Unit

Conventional processes for making p-xylene typically does not include co-feeding toluene to a VPI process. Thus, the only feed supplied to the VPI process in a conventional process is a p-xylene-depleted raffinate stream exiting the p-xylene separation/recovery system, which typically consists essentially of m-xylene, o-xylene, EB, p-xylene, and toluene at a toluene concentration lower than 2 wt % (e.g., no higher than 1 wt %), based on the total weight of the raffinate stream. In the VPI unit, an isomerization catalyst system is disposed. Under temperature and pressure conditions such that at least a portion, preferably substantially all, of the materials in the VPI are present in vapor phase. In contacting the first catalyst system, the xylene molecules undergo isomerization reactions to produce a xylenes mixture having a composition closer to the equilibrium mixture. Because the raffinate feed supplied to the VPI unit tends to comprise p-xylene substantially lower than its equilibrium concentration, a portion of the m-xylene and/or o-xylene molecules isomerize to form p-xylene in the first product mixture effluent. In addition, the EB molecules contained in the feed supplied to the VPI unit is are desirably converted into other products, e.g., benzene, via e.g., de-ethylation catalyzed by the first catalyst in the VPI unit. Without intending to be bound by a particular theory, it is believed to be highly desirable that de-ethylation occurs in the channel of a shape-selective zeolite material in the first catalyst system which would preferentially allow EB molecules and smaller size molecules to enter. While m-xylene and o-xylene have substantially larger molecular sizes than EB and therefore would diffuse into the channel at a much lower rate than EB, p-xylene can enter the channel at a high rate due to a similar molecular dimension to EB. Inside the channel, de-methylation, transalkylation, and disproportionation of p-xylene can occur, leading to the formation of C9 aromatics, toluene and methane, and resulting in a net xylenes loss in the VPI isomerization process of a conventional VPI process. Such xylenes loss can be particularly pronounced where the raffinate feed supplied to the VPI unit comprises p-xylene at a high concentration, e.g., ≥3.0 wt %, ≥4.0 wt %, or ≥5.0 wt %, based on the total weight of the feed to the VPI unit. It would be highly desirable to reduce the xylenes loss in the VPI unit.

In the process/system of this disclosure, to reduce xylenes loss in a VPI unit, additional toluene is co-fed into the VPI unit such that the total toluene concentration in the total feed supplied to the VPI unit, including the toluene contained in the raffinate (or the first C8 aromatics mixture), is at least 2 wt %, based on the total weight of the aromatic feed mixture supplied to the VPI. It has been found that such co-feeding of toluene has unexpectedly improve xylenes loss in the VPI unit. Without intending to be bound by a particular theory, it is believed that the co-fed toluene, due to its similar molecular dimension (at least in one direction) to p-xylene, competes against p-xylene in entering the channels in the catalyst responsible for dealkylation reactions, thereby reducing the amount of p-xylene entering the dealkylation channels, hence reducing dealkylation of p-xylene and xylenes loss. Furthermore, the reactions of xylenes loss through disproportionation and/or transalkylation would produce toluene and C9 aromatic hydrocarbons. Thus, by increasing the toluene concentration in the reaction mixture in the VPI, the chemical equilibriums of xylenes loss reactions are shifted toward xylenes production, thereby suppressing xylenes loss. Still further, toluene may undergo disproportionation in the presence of the isomerization catalyst system, leading to the formation of benzene and xylenes, further reducing xylenes loss in the isomerization reactor. Dealkylation, transalkylation, and disproportionation of toluene result in the production of benzene, which can be separated as a valuable product.

The first C8 aromatics mixture fed to the VPI unit, which can be a portion of a raffinate from a p-xylene separation/recovery system, can comprise p-xylene at various concentration of C(pX) ranging from c1 to c2 wt %, based on the total weight of the C8 aromatics present in the first C8 aromatics mixture, wherein c1 and c2 can be, independently, e.g., 0.5, 1, 2, 3, 4, 5, 6, 7, 8, 9, 10, 11, 12, 13, 14, 15, 16, 17, 18, 19, 20, as long as c1<c2. Typically C(pX) is lower than the p-xylene concentration in an equilibrium mixture consisting of p-xylene, m-xylene, and o-xylene at the same temperature. Preferably C(pX)≤15. More preferably C(pX)≤10. Still more preferably C(pX)≤8. As mentioned above, the higher the C(pX) in the first C8 aromatics mixture, the more significant xylenes loss can be in a VPI process, and the more advantageous the process of this disclosure comprising co-feeding toluene to the VPI unit is. The xylenes loss reduction of the process of this disclosure compared to a comparative process without co-feeding toluene can be very significant, as described below, especially where C(pX)≥5, and even where 3≤C(pX)≤5.

The first C8 aromatics mixture can comprise m-xylene at various concentration of C(mX) ranging from m1 to m2 wt %, based on the total weight of the C8 aromatics present in the first C8 aromatics mixture, wherein m1 and m2 can be, independently, e.g., 20, 25, 30, 35, 40, 45, 50, 55, 60, 65, 70, 75, or 80, as long as m1<m2. C(mX) can be significantly higher than the m-xylene concentration in an equilibrium mixture consisting of p-xylene, m-xylene, and o-xylene at the same temperature, especially if the first C8 aromatics mixture consists essentially of xylenes only and is substantially free of EB.

The first C8 aromatics mixture can comprise o-xylene at various concentration of C(oX) ranging from n1 to n2 wt %, based on the total weight of the C8 aromatics present in the first C8 aromatics mixture, wherein n1 and n2 can be, independently, e.g., 10, 15, 20, 25, 30, 35, 40, 45, 50, as long as n1<n2. C(oX) can be significantly higher than the o-xylene concentration in an equilibrium mixture consisting of p-xylene, m-xylene, and o-xylene at the same temperature, especially if the first C8 aromatics mixture consists essentially of xylenes only and is substantially free of EB.

Among all xylenes present in the first C8 aromatics mixture, m-xylene and o-xylene can be present at any ratio. Thus, the ratio of m-xylene to o-xylene can range from r1 to r2, where r1 and r2 can be, independently, e.g., 0.1, 0.2, 0.3, 0.4, 0.5, 0.6, 0.7, 0.8, 0.9, 1, 2, 3, 4, 5, 6, 7, 8, 9, 10, as long as r1<r2.

The first C8 aromatics mixture fed to the VPI unit can comprise xylenes in total at a concentration of C(aX) wt % ranging from c3 to c4 wt %, based on the total weight of the first C8 aromatics mixture, wherein c3 and c4 can be, independently, e.g., 50, 55, 60, 65, 70, 75, 80, 85, 90, 91, 92, 93, 94, 95, 96, 97, 98, or 99, as long as c3<c4. The first C8 aromatics mixture can consist essentially of xylenes and EB.

The first C8 aromatics mixture can comprise EB at a concentration of C(EB) wt % ranging from c5 to c6 wt %, based on the total weight of the first C8 aromatics mixture, wherein c5 and c6 can be, independently, e.g., 1, 2, 3, 4, 5, 6, 7, 8, 9, 10, 12, 14, 15, 16, 18, 20, 22, 24, 25, 26, 28, 30, as long as c5<c6. Preferably 2≤C(EB)≤25. More preferably 3≤C(EB)≤20. Still more preferably 5≤C(EB)≤15.

The first C8 aromatics mixture comprise C8 aromatic hydrocarbons (i.e., the xylenes and EB) at an aggregate concentration of, e.g., ≥90, or ≥92, or ≥94, or ≥95, or ≥96, or ≥98, or even ≥99 wt %, based on the total weight of the first C8 aromatics mixture.

The first C8 aromatics mixture, depending on its source (e.g., a xylenes distillation column), may comprise toluene at various amounts, but typically no greater than 2 wt %, based on the total weight of the first C8 aromatics mixture. In certain exemplary first C8 aromatics mixture, the concentration of toluene is approximately 0.9 wt %, based on the total weight of the first C8 aromatics mixture. Any additional toluene co-fed into the VPI unit along with the first C8 aromatics mixture can help with xylenes loss reduction in the VPI compared to a conventional process without co-feeding toluene. However, in the process of this disclosure, to produce an appreciable xylenes loss reduction, sufficient quantity of toluene is co-fed into the VPI unit such that the aggregate quantity of toluene fed into the VPI unit, including the co-fed toluene and any toluene contained in the first C8 aromatics mixture, is ≥2 wt % of the total weight of the toluene and the first C8 aromatics mixture, which can be from y1 to y2 wt %, where y1 and y2 can be, independently, e.g.: 2, 3, 4, 5, 6, 7, 8, 9, 10, 12, 14, 15, 16, 18, 20, as long as y1<y2. At higher amount of co-fed toluene, the xylenes loss reduction can be greater. At very high amount of co-fed toluene, xylenes loss can be minimized Above a certain threshold quantity of co-fed toluene, increasing the quantity of co-fed toluene to the VPI unit may not further reduce xylenes loss.

However, in certain situations, it may be highly desirable to co-feed large quantity of toluene into the VPI unit above the threshold quantity. For example, where the first C8 aromatics mixture supplied to the VPI unit is below the design operating capacity of the VPI unit, it would be highly advantageous to co-feed a substantial quantity of toluene into the VPI unit, so that the unit is operated at a desired WHSV, even if the quantity of co-fed toluene is well above the threshold quantity. Operating the VPI unit at desired WHSV, especially close to design WHSV, can eliminate the need for costly modification of the VPI unit and its peripheral equipment, including the feed and effluent heat exchangers, and the gas compressors. Once the co-feed is established, the toluene can be internally recycled such that no external source is required.

As discussed above, it is believed that the xylenes loss reduction effect of co-feeding toluene to the VPI unit is at least partly due to the increased completion from toluene against p-xylene in the de-ethylation channel in the zeolite used in the first catalyst system in the VPI unit. Thus, to achieve significant reduction of xylenes loss, it is highly desired in certain embodiments that the molar ratio (R1) of the total quantity of toluene fed into the VPI unit, including the additional, co-fed toluene and any toluene contained in the first C8 aromatics mixture, to the quantity of p-xylene in the first C8 aromatics mixture be in a range from R1a to Rib, where R1a and R1b can be, independently, e.g., 0.40, 0.50, 0.75, 1.0, 1.2, 1.4, 1.5, 1.6, 1.8, 2.0, 2.2, 2.4, or 2.5, as long as R1a<R1b. At a molar ratio R1 of 0.40, the significant amount of toluene relative to p-xylene reduces the amount of p-xylene entering the zeolite de-ethylation channels to reduce xylenes loss. The higher the molar ratio R1 is, the lower the chance of p-xylenes molecules entering into the de-ethylation channels. At R1>2.5, the additional xylenes loss reduction can be minimal by increasing the quantity of co-fed toluene.

As non-limiting examples of toluene that may be co-fed into the VPI unit, mention can be made of toluene from a reformate splitter, toluene from a benzene column, “crude” toluene from a p-xylene separation section, by-product toluene from a VPI unit or LPI unit, or toluene produced in a transalkylation unit. Preferably, such toluene is produced on the same general production facility. Alternatively, toluene from other sources may be used.

Xylenes loss in a process of this disclosure (“Lx(1)”) can be calculated as Lx(1)=100%*(W1−W2)/W1, where W1 is the aggregate weight of all xylenes present in the first C8 aromatics mixture, and W2 is the aggregate weight of all xylenes present in the first product mixture effluent. In a comparative, conventional process otherwise identical to such process of this disclosure except no additional toluene is supplied to the VPI unit, and the C8 aromatics mixture comprise 0.9 wt % of toluene based on the total weight of the C8 aromatics mixture, xylenes loss (“Lx(2)”) can be calculated as Lx(2)=100%*(W3-W4)/W3, where W3 is the aggregate weight of all xylenes present in the first C8 aromatics mixture, and W4 is the aggregate weight of all xylenes present in the first comparative product mixture effluent produced from the comparative process. The difference between Lx(2) and Lx(1) (i.e., Lx(2)−Lx(1)) in certain embodiments can be in a range from d1 to d2%, where d1 and d2 can be, independently, 0.2, 0.3, 0.4, 0.5, 0.6, 0.7, 0.8, 0.9, 1.0, 1.1, 1.2, 1.3, 1.4, 1.5, as long as d1<d2. For a VPI unit with a substantial work load, such xylenes loss reduction can translate to substantial savings over a long period of operation. Therefore, clearly, compared to a convention VPI process without feeding an additional quantity of toluene to the VPI unit, a highly similar process with a quantity of additional toluene fed into the VPI can achieve substantial xylenes loss reduction and significant product yield improvement.

The first product mixture effluent from the VPI unit is depleted in EB compared to the first C8 aromatics feed fed into the VPI. The EB concentration in the total feed supplied to the VPI unit can be recorded as C(EB1). The EB concentration in the first product mixture effluent from the VPI unit can be recorded as C(EB2). In the process of this disclosure, the ratio of C(EB1)/C(EB2) can range from r3 to r4, where r3 and r4 can be, independently, e.g., 0.1, 0.2, 0.3, 0.4, 0.5, 0.6, 0.7, or 0.8, as long as r3<r4. Thus, a significant portion of the EB in the feed to the VPI unit is converted into other molecules.

The first product mixture effluent from the VPI unit comprises p-xylene at a concentration higher than the first C8 aromatics mixture, thanks to the isomerization reactions resulting in the conversion and m-xylene and/or o-xylene to p-xylene. The first product mixture effluent further comprises toluene, light hydrocarbons generated from, e.g., EB dealkylation, and possibly aliphatic hydrocarbon byproducts due to side reactions. While it may be possible to directly recycle at least a portion (or the entirety) of the first product mixture effluent to a p-xylene separation/recovery system to recover the p-xylene at an enhanced concentration, in certain embodiments, it is desired to separate at least a portion (or the entirety) of the first product mixture effluent to remove at least a portion of the light hydrocarbons, at least a portion of the aliphatic hydrocarbons (e.g., in a deheptanizer), at least a portion of the toluene (e.g., in a xylenes distillation tower), to obtain an aromatic hydrocarbon mixture rich in C8 aromatics, and particularly p-xylene, which is then supplied to a p-xylene separation/recovery system to produce a high-purity p-xylene product.

The isomerization/EB conversion processes in this disclosure may be operated at high EB conversion levels, such as from c(EB)1 to c(EB)2, where c(EB)1 and c(EB)2 can be, independently, e.g., 50, 55, 60, 65, 70, 75, 80, 85, 90, 95, 98, 99, or even 100, as long as c(EB)1<c(EB)2. Xylenes isomerization and EB conversion can be accomplished using, e.g., a dual-bed reactor, in which the EB conversion may take place in a first bed of the first catalyst system and the xylene isomerization may take place in a second bed of the catalyst system, or a single bed reactor, in which EB conversion and xylenes isomerization occur in contact with a single at bed of catalyst.

In the EB conversion process of the process of this disclosure, in at least one embodiment, an EB selectivity to benzene can range from s1% to s2%, where s1 and s2 can be, independently, e.g.: 60, 65, 70, 75, 80, 85, 90, 95, 98, or even 99, as long as s1<s2. For example, injection of 10 wt % of toluene co-feed in a VPI unit may provide a reduction of xylenes loss (only 1.75% of xylenes loss at 75% EB conversion) and an increase in EB selectivity to benzene (e.g., 89% selectivity at 75% EB conversion). The increased selectivity for benzene in EB conversion in combination with a significant xylenes loss reduction is totally unexpected. The benzene can be recovered for sale or hydrogenation to produce cyclohexane or can be fed to one or more units of the apparatus.

### IV. The Process in Relation to a VPI Process in a VPI Unit and a LPI Process in a LPI Unit

As described earlier in this disclosure, it is highly advantageous to deploy a LPI unit in a p-xylene production facility in lieu of or in addition to a VPI unit. It is highly advantageously to add a LPI unit to a p-xylene production facility having only VPI unit(s). The LPI process has many advantages over the VPI process. For example, the isomerized effluent from a LPI unit may be directly recycled back in its entirety to a p-xylene separation/recovery system because of the low concentration of light hydrocarbons and aliphatic hydrocarbons present in the LPI isomerized effluent resulting from the low operating temperature and lack of side reactions that could occur in a VPI process operated at a much higher temperature.

Thus, in certain embodiments of this disclosure, it is highly advantageous to supply a second C8 aromatics mixture into a LPI unit, operate the LPI under conditions effective to isomerize m-xylene and/or o-xylene in the presence of a second catalyst system in the LPI unit, and obtain a second product mixture effluent from the LPI unit, the second product mixture effluent being p-xylene rich relative to the second C8 aromatics mixture.

It is contemplated that the LPI unit and the VPI unit can be operated in series, i.e., at least a portion of the C8 aromatics mixture supplied to one of them is at least a portion of the product mixture effluent produced from the other. Thus, a VPI unit may be placed upstream of an LPI unit. In such case, the first C8 aromatics mixture comprising EB and xylenes, as well as co-feed toluene are supplied into the VPI unit, where the xylenes undergo isomerization reactions, and the EB is at least partly converted into other products such as benzene. The first product mixture effluent from the VPI unit, depleted in EB and partially enriched in p-xylene compared to the first C8 aromatics mixture, is at least partly supplied to the LPI unit, where additional isomerization reactions of the xylenes occur in the presence of a second catalyst system in the LPI unit. The second product mixture effluent from the LPI can then be separated to remove light hydrocarbons and aliphatic hydrocarbons produced primarily in the VPI unit, to obtain a p-xylene-rich C8 aromatics mixture, which can be supplied to a p-xylene separation/recovery system to produce a high-purity p-xylene product. It is also possible to place the LPI unit upstream of the VPI unit.

In one particularly desirable embodiment, the LPI unit and VPI unit are configured to operate in parallel, i.e., the first C8 aromatics mixture supplied to the VPI unit is not directly received from, partly or in its entirety, from the second product mixture effluent produced from the LPI unit, and vice versa. Thus, the first C8 aromatics mixture and the second C8 aromatics mixture may be supplied from the same source, e.g., the same p-xylene-depleted raffinate stream produced from a p-xylene separation/recovery system. In such case the first and second aromatics mixtures can have substantially the same composition. Alternatively, the first C8 aromatics mixture and the second aromatics mixture may be supplied from different sources. For example, the first C8 aromatics mixture supplied to the VPI unit may be sourced from a C8 distillation column, which in turn, receives most of the C8 aromatics from a catalytic naphtha reformer, which can comprise EB at high concentrations; and the second C8 aromatics mixture supplied to the LPI unit may be sourced from a toluene disproportionation process or toluene/benzene methylation with methanol process, which can contain EB at very low concentrations. The VPI process can be more effective than the LPI process in converting EB.

Nonetheless, due to the advantages of an LPI process over a VPI process, in a xylene production facility with a given amount of C8 aromatics mixture subject to isomerization, it is highly desirable to supply a significant portion of the C8 aromatics mixture to the LPI unit, as long as a LPI unit and a VPI unit are both present in the facility, especially if they are operated in parallel. To that end, in such embodiments of this disclosure, if the weight of the first C8 aromatics mixture fed to the VPI unit is Q1, the weight of the second C8 aromatics mixture fed to the LPI unit is Q2, the ratio of Q2/Q1 is R3, it is highly advantageous that R3≥0.25. Thus, R3 can range from R3a to R3b, where R3a and R3b can be, independently, e.g.: 0.25, 0.30, 0.35, 0.45, 0.50, 0.60, 0.70, 0.80, 0.90, 1.0, 1.5, 2.0, 2.5, 3.0, 3.5, 4.0, 4.5, or 5.0, as long as R3a<R3b.

The addition of an additional LPI unit to a xylenes production facility having a pre-installed VPI unit can divert a portion of the originally planned C8 aromatics mixture subject to isomerization for the VPI unit only to the LPI unit, which can result in the VPI unit operating under its original design capacity if no additional feed is supplied to the VPI unit. In such case, as discussed earlier in this disclosure, it would be highly advantageous to co-feed toluene to the VPI unit pursuant to the process of this disclosure, so that the VPI unit is operated in desired WHSV range. In such cases, the co-fed toluene to the VPI unit can be at substantial quantity, even well above the threshold level as discussed earlier in this disclosure. In such cases, the total weight of the toluene fed to the VPI unit, including any toluene present in the first C8 aromatics mixture, can be ≥3% (or ≥5%, or ≥8%, or ≥10%, or ≥15%, or ≥18%, and ≤20%) of the weight of first C8 aromatics mixture fed into the VPI unit when the VPI unit is operated at full capacity where toluene constitutes no more than 1 wt % of the total weight of the first C8 aromatics mixture and toluene fed into the VPI unit.

Therefore, the process of this disclosure including a VPI process with toluene co-feeding and a LPI process can achieve an overall high energy efficiency, less need to separate byproducts produced from the VPI process, and long operating cycles for both the VPI and LPI units.

Monitoring p-Xylene Concentration and Adjusting Toluene Co-feed Quantity

Another aspect of this disclosure relates to a process for producing p-xylene, the process comprise a step of determining a concentration of p-xylene (“C(pX)”) in the first C8 aromatics mixture supplied to the VPI unit, and supplying additional co-feed toluene to the VPI unit such that the total weight of toluene fed into the VPI unit, including toluene contained in the first aromatics mixture, if any, is at least 2 wt % of the total weight of the C8 aromatics mixture and the toluene fed into the VPI unit.

The p-xylene concentration in the first C8 aromatics mixture can fluctuate. Offline instruments, or inline instruments connected with the supply line of the first C8 aromatics mixture, may be used to determine the p-xylene concentration therein. Such instruments can include, e.g., gas chromatograph (“GC”), mass spectrometry (“MS”), GC-MS, FT-IR spectrometer, and the like. If the C(pX) is high, e.g., ≥5 wt %, based on the total weight of the first C8 aromatics mixture, additional toluene can be co-fed into the VPI unit to reduce xylenes loss in the VPI. Where 3≤C(pX)<5 wt %, one may also opt to co-feed toluene to the VPI unit, even though the xylenes loss reduction would not be as much need as where C(pX)≥5 wt %.

As described above, to achieve a high degree of xylenes loss reduction, a significant quantity of toluene relative to the p-xylene in the first C8 aromatics feed is highly desired. Thus, the ratio of the moles of toluene fed into the VPI unit to the moles of the p-xylene contained in the first C8 aromatics mixture fed into the VPI unit (ratio R1), can be in a range from R1a to R1b, where R1a and R1b can be, independently, e.g., 0.40, 0.50, 0.75, 1.0, 1.2, 1.4, 1.5, 1.6, 1.8, 2.0, 2.2, 2.4, or 2.5, as long as R1a<R1b. The weight of the total toluene fed to the VPI unit, including any toluene present in the first aromatics mixture, can be in a range from y1 to y2 wt %, based on the total weight of the first C8 aromatics feed and the co-feed toluene, where y1 and y2 can be, independently, e.g.: 2, 3, 4, 5, 6, 7, 8, 9, 10, 12, 14, 15, 16, 18, 20, as long as y1<y2.

V. Exemplary p-Xylene Production System

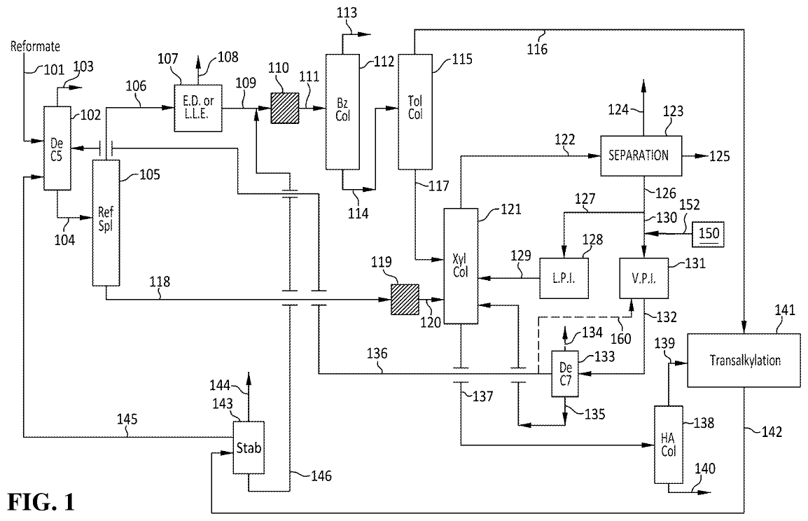

FIG. 1 is a schematic flow diagram for producing p-xylene and other hydrocarbon products from a reformate stream produced from a catalytic reformer in an oil refinery. In FIG. 1, a catalytic reformate feed stream containing aromatics is supplied by line 101 to a depentanizer 102 to remove the C5-fraction. Pentane and lighter hydrocarbons are removed via line 103, while the C6+ bottoms fraction is fed via line 104 to a reformate splitter 105.

The C7-containing stream from the reformate splitter 105 is sent via line 106 to an extractive distillation or liquid-liquid extraction unit 107, where aliphatic C6, C7, and C8 hydrocarbons are removed via line 108 to leave a benzene- and toluene-enriched stream, which is fed via line 109 to an olefins saturation zone 110. The olefins saturation zone 110 may be a clay treater or any other means effective to remove olefins contaminants in aromatic streams, including catalytic processes, with optional hydrogen addition. The effluent from the olefins saturation zone 110 is fed via line 111 to a distillation column 112 from which C6 aromatics product (benzene) is collected via line 113. A bottoms stream from column 112 is sent via line 114 to a distillation column 115. An overhead toluene stream is produced from column 115 and transferred via line 116 to a transalkylation unit 141 and a C8+ bottoms fraction is fed via line 117 to a xylenes distillation column 121.

The C8 aromatic hydrocarbon-containing stream recovered from the reformate splitter 105 is supplied by line 118 to an olefins saturation zone 119. The olefins saturation zone 119 may be a clay treater or any other means effective to remove olefins contaminants in aromatic streams, including catalytic processes, with optional hydrogen addition. The effluent from the olefins saturation zone 119 is fed via line 120 to the xylenes distillation column 120. In at least one embodiment, the effluent from the olefins saturation zone 119 is supplied to the column 121 separately from and above the supply point for the C8+ fraction in line 117 since the stream in line 120 is much lighter than the stream in line 117.

The xylenes distillation column 121 produces an overhead C9+-depleted stream (i.e., a C8-rich aromatics stream) from the feeds to the distillation column 121. This overhead stream is then supplied via line 122 to a p-xylene separation section 123, where p-xylene can be conventionally separated by adsorption or crystallization or a combination of both and recovered via line 124.

When p-xylene is separated by adsorption (of separation section 123), the adsorbent used preferably contains a zeolite. Typical adsorbents used include crystalline alumino-silicate zeolites either natural or synthetic, such as zeolite X or Y or mixtures thereof. These zeolites are preferably exchanged by cations such as alkali or alkaline earth or rare earth cations. The adsorption column is preferably a simulated moving bed column (SMB) and a desorbent is used such as paradiethylbenzene, paradifluorobenzene, diethylbenzene or toluene or mixtures thereof.

Residual toluene in the overhead stream from the xylene splitter column 121 is removed from the p-xylene separation section 123 via line 125, while the remaining p-xylene-depleted stream 126 (the filtrate from the crystallizer or raffinate from adsorption separation system) is sent toward a liquid phase isomerization (LPI) unit 128 and a vapor phase isomerization (VPI) unit 131. For example, a first portion 130 of the p-xylene-depleted stream is fed to VPI unit 131, and a second portion 127 of the p-xylene-depleted stream is fed to LPI unit 128. The first portion 130 is a first C8 aromatics mixture that can preferably comprise xylenes and EB at a total concentration of, e.g., at least 90 wt %, more preferably at least 92 wt %, still more preferably 95 wt %, and still more preferably 98 wt %, based on the total weight of the first C8 aromatics mixture. Typically, the first portion 130 can comprise toluene at, e.g., below 2 wt %, based on the total weight of the first C8 aromatics mixture, preferably no greater than 1.5 wt %, still more preferably no greater than 1.0 wt %, still more preferably no greater than 0.9 wt %. The first portion 130 can comprise EB at a concentration of, e.g., at least 3 wt %, based on the total weight of the first C8 aromatics mixture, such as at least 10 wt %, such as at least 25 wt %. The first portion 130 can comprise p-xylene at a concentration of, e.g., at least 0.5 wt %, based on the total weight of the first C8 aromatics mixture, such as at least 5 wt %, or at least 10 wt %. The first portion 130 can comprise o-xylene at a concentration of, e.g., at least 10 wt %, based on the total weight of the first C8 aromatics mixture, such as at least 25 wt %, such as at least 50 wt %. The first portion 130 can comprise m-xylene at a concentration of at least 20 wt %, based on the total weight of the first C8 aromatics mixture, such as at least 40 wt %, such as at least 70 wt %.

The second portion 127 is a second C8 aromatics mixture that can preferably comprise xylenes and EB at a total concentration of at least 90 wt %, more preferably at least 92 wt %, still more preferably 95 wt %, and still more preferably 98 wt %, based on the total weight of the second C8 aromatics mixture. E.g., the second portion 127 can comprise toluene at below 2 wt %, based on the total weight of the second C8 aromatics mixture, preferably no greater than 1.5 wt %, still more preferably no greater than 1.0 wt %, still more preferably no greater than 0.9 wt %. The second portion 127 can comprise EB at a concentration of at least 3 wt %, based on the total weight of the second C8 aromatics mixture, such as at least 10 wt %, such as at least 25 wt %. The second portion 127 can comprise p-xylene at a concentration of e.g., at least 0.5 wt %, based on the total weight of the second C8 aromatics mixture, such as at least 5 wt %, such as at least 10 wt %. The second portion 127 can comprise o-xylene at a concentration of at least 10 wt %, based on the total weight of the second C8 aromatics mixture, such as at least 25 wt %, such as at least 50 wt %. The second portion 127 can comprise m-xylene at a concentration of, e.g., at least 20 wt %, based on the total weight of the second C8 aromatics mixture, such as at least 40 wt %, such as at least 70 wt %. In at least one embodiment, the second C8 aromatics mixture has substantially the same composition as the first C8 aromatics mixture. As shown in FIG. 1, the first portion 130 and the second portion 127 are split from the same effluent derived from the same p-xylene separation/recovery unit, and therefore have substantially the same composition. However, it is also contemplated that the LPI unit and the VPI unit may be configured to receive p-xylene-depleted streams derived from different p-xylene separation/recovery units having different compositions. For example, the LPI unit can be configured to receive primary a p-xylene-depleted raffinate stream from a crystallizer, and the VPI unit may be configured to receive primary a p-xylene-depleted raffinate stream from an adsorption chromatography separation unit.

The weight of the first C8 aromatics mixture fed to the VPI unit can be referred to as Q1, and the weight of the second C8 aromatics mixture fed to the LPI can be referred to as Q2. The ratio of Q2/Q1 can be referred to as R3, and R3 can be ≥0.25, e.g., R3≥0.40, or R3≥0.50.

As discussed above, conventional C8 aromatics isomerization processes do not include co-feeding toluene to a VPI unit, and therefore can result in high xylenes loss in the VPI process, especially if the first aromatics mixture comprises p-xylene at a high concentration.

FIG. 1 shows an exemplary process/system of this disclosure which includes co-feeding additional amount of toluene from a toluene source 150 coupled (directly or indirectly) with VPI unit 131 to the VPI unit 131. Toluene source 150 can be coupled directly with VPI unit 131. Additionally or alternatively, the toluene source 150 can be coupled with line 126 exiting the separation unit or an introduction feed line coupled with the VPI unit to provide introduction of the co-feed (via line 152) to the line(s) and/or isomerization unit. Any amount of additional toluene can help reducing p-xylene dealkylation and xylenes loss. To produce significant reduction of xylenes loss, it is highly desirable that the total quantity of toluene fed into the VPI unit, including the toluene contained in the first C8 aromatics mixture (feed in line 130), if any, is substantial compared to the total quantity of p-xylene in the first C8 aromatics mixture fed into the VPI unit. Thus, where the molar ratio of toluene (including the toluene co-fed to the VPI unit and the toluene contained in the first C8 aromatics mixture) to the moles of p-xylene contained in the first C8 aromatics mixture is R1, it may be highly desirable R1≥0.40, such as ≥0.50, ≥0.60, ≥0.70, ≥0.80, ≥0.90, ≥1.0, ≥1.50, or ≥2.0. To reduce the quantity of toluene in the first product mixture effluent exiting the VPI unit, which would require separation and possible recycling, it may be desirable R1≤2.5, such as R1≤2.0, R1≤1.5, R1≤1.0, depending on the needs of the particular first C8 aromatics mixture fed to the VPI. Thus, it is possible 0.40≤R1≤2.5, or 0.5≤R1≤2.5, and R1 may vary within many other ranges. Alternatively, the weight of toluene fed to the VPI unit, including any toluene contained in the first C8 aromatics mixture, can be at least 3 wt % of the total weight of the C8 aromatics mixture and the toluene fed into the VPI unit, such as at least 5 wt %, such as from 3 wt % to 20 wt %, such as from 5 wt % to 15 wt %, such as 10 wt %. The toluene source 150 can be a toluene storage tank, or a vessel producing a toluene stream in the same facility, e.g., the toluene distillation column 115. In such case, a portion of the toluene carried in line 116 can be diverted and fed into the VPI unit.

In at least one embodiment, the toluene co-feed is fed to the VPI unit or to a line coupled with the VPI unit such that the weight of toluene fed is at least 3% of the weight of the first C8 aromatics mixture fed into the VPI unit when the VPI unit is operated at full capacity where toluene constitutes no more than 1 wt % of the total weight of the first C8 aromatics mixture and toluene fed into the VPI unit.

The isomerization process of this disclosure can exhibit a first xylenes loss of Lx(1), where Lx(1)=100%*(W1−W2)/W1, W1 is the aggregate weight of all xylenes present in the first C8 aromatics mixture, W2 is the aggregate weight of all xylenes present in the first product mixture effluent. A comparative process can exhibit a second xylenes loss of Lx(2) where no additional toluene is supplied to the VPI unit, the C8 aromatics mixture comprises 0.9 wt % of toluene based on the total weight of the C8 aromatics mixture, and all other conditions are held equal to the preceding process of this disclosure, Lx(2)=100%*(W3−W4)/W3, W3 is the aggregate weight of all xylenes present in the first C8 aromatics mixture, and W4 is the aggregate weight of all xylenes present in the first comparative product mixture effluent produced from the comparative process. The process of this disclosure can achieve a significant xylenes loss reduction compared to the comparative process of Lx(2)−Lx(1), e.g., Lx(2)−Lx(1)≥0.2, such as 0.2%≤Lx(2)−Lx(1)≤1.5%.

The effluent from VPI unit 131 may comprise EB at a concentration of at least 1 wt %, based on the total weight of the effluent, such as at least 5 wt %, such as at least 10 wt %. The effluent from VPI unit 131 can comprise p-xylene at a concentration of at least 15 wt %, based on the total weight of the C8 aromatics in the first product mixture effluent, e.g., ≥16, ≥18, ≥20, ≥22, ≥24 wt %. The effluent from VPI unit 131 may comprise toluene at a concentration of at least 2 wt %, based on the total weight of the effluent, such as at least 3 wt %, or at least 5 wt %, or at least 8 wt %, or at least 10 wt %, as the case may be depending on the needs of the VPI unit. The effluent from VPI unit 131 may comprise o-xylene at a concentration of at least 5 wt %, based on the total weight of all xylenes in the effluent, .g., ≥16, 18, 20, 22, 24 wt %. The effluent from VPI unit 131 may comprise m-xylene at a concentration of at least 10 wt %, based on the total weight of all xylenes in the effluent, such as at least 30 wt %, such as at least 50 wt %. In general, where the feed to the VPI unit comprises a p-xylene depleted raffinate from a p-xylene recovery unit having a p-xylene concentration below 15 wt %, the VPI effluent can contain p-xylene at a concentration up to its equilibrium concentration of about 25 w %. The effluent from VPI unit 131 may comprise benzene at a concentration of at least 0.5 wt %, based on the total weight of the effluent, such as at least 1 wt %, such as at least 2 wt %. It is desirable that benzene concentration in the VPI effluent not exceed, e.g., 5 wt %, such as ≤4 wt %, ≤3 wt %, or ≤2 wt %, based on the total weight of the VPI effluent.

Meanwhile, in the LPI unit 128, xylene isomerization is preferably achieved in the liquid phase. Any suitable liquid phase catalytic isomerization process known to those skilled in the art can be used to effect the xylene isomerization in LPI unit 128, but one preferred catalytic system employs an intermediate pore size zeolite.

Any suitable liquid phase catalytic isomerization process can be used in the liquid phase xylenes isomerization unit, such as a catalytic system described in U.S. Patent Application Publication Nos. 2011/0263918 and 2011/0319688, the contents of each of which are incorporated herein by reference. The xylene isomerization conditions employed in the LPI unit 128 are selected so as to isomerize xylenes in the p-xylene depleted stream, and thereby produce an isomerized stream having a higher concentration of p-xylene than the p-xylene depleted stream. Exemplary conditions include a temperature of from about 220° C. to 480° C. (alternatively from about 220° C. to about 350° C., alternatively from about 350° C. to 480° C.), a pressure from about 446 kPa to about 3,500 kPa (alternatively from about 1,300 kPa to about 2,860 kPa), a WHSV of from about 0.5 hr-1 to about 50 hr-1 (such as from about 3 hr-1 to about 50 hr⁻¹) and a hydrogen to hydrocarbon molar ratio from about 0.7 to about 5. The WHSV is based on the weight of catalyst composition, i.e., the total weight of active catalyst and, if used, binder therefor.

The effluent from the LPI unit 128 is supplied via line 129 to the xylene column 121. The effluent from LPI unit 128 may contain close to equilibrium p-xylene (such as about 24% p-xylene, based on all xylenes present in the LPI unit effluent).

The effluent from the VPI unit 131 is supplied via line 132 to the deheptanizer 133, from which off gas (e.g., light hydrocarbons such as ethane and/or propane) is removed via line 134, a C6/C7 stream (which can be a toluene-rich fraction) is sent to the depentanizer 102 via line 136, and a C8+ aromatics effluent is fed via line 135 to xylene column 121.

The C9+ aromatic hydrocarbon-containing stream recovered from xylene column 121 is supplied via line 137 to a heavy aromatics distillation column 138. The heavy aromatics distillation column 138 separates the C9+ aromatic hydrocarbons supplied by line 137 into a C9/C10/light C11-containing fraction which is removed in line 139 and C11+-containing fraction which is supplied to the gasoline pool, a fuel oil pool, or to a topping column via line 140 (which contains C11+ compounds). The C9/C10/light C11-containing fraction 139 is then fed to the transalkylation unit 141 in combination with the toluene-enriched streams supplied via line 116.

Any transalkylation process known to those skilled in the art can be used, but one preferred process employs the multi-stage catalytic system described in U.S. Pat. No. 7,663,010, incorporated herein by reference. Such a system comprises (i) a first catalyst comprising a first molecular sieve having a Constraint Index in the range of 3-12 and containing 0.01 to 5 wt % of at least one source of a first metal element of Groups 6-10 of the Periodic Table and (ii) a second catalyst comprising a second molecular sieve having a Constraint Index less than 3 and comprising 0 to 5 wt % of at least one source of a second metal element of Groups 6-10 of the Periodic Table, wherein the weight ratio of the first catalyst or the second catalyst is in the range of 5:95 to 75:25 and wherein the first catalyst is located upstream of the second catalyst.

Examples of suitable molecular sieves having a Constraint Index of 3-12 for use in the first catalyst include ZSM-5, ZSM-11, ZSM-22, ZSM-23, ZSM-35, ZSM-48, ZSM-57, and ZSM-58, with ZSM-5 being preferred. Examples of suitable molecular sieves having a Constraint Index of less than 3 for use in the second catalyst include zeolite beta, zeolite Y, Ultrastable Y (USY), Dealuminized Y (Deal Y), mordenite, ZSM-3, ZSM-4, ZSM-12, ZSM-18, NU-87, and ZSM-20, with ZSM-12 being preferred. Specific examples of useful metals for each of the first and second catalysts include iron, ruthenium, osmium, nickel, cobalt, rhenium, molybdenum, tin, and noble metals such as platinum, rhodium, iridium or palladium.

The transalkylation process can be conducted in any appropriate reactor including a radial flow, fixed bed, continuous down flow or fluid bed reactor. The conditions in the first and second catalyst bed can be the same or different but generally can comprise a temperature from, e.g., 100° C. to 1000° C., preferably in the range of 300 to 500° C.; a pressure in the range of, e.g., 790 to 7000 kPa-a (kilo-Pascal absolute), preferably in the range of 2170 to 3000 kPa-a; a hydrogen to hydrocarbon molar ratio from, e.g., 0.01 to 20, preferably from 1 to 10; and a WHSV from 0.01 to 100 hr⁻¹, preferably in the range of 1-20 hr⁻¹.

The effluent from the transalkylation unit 141 is fed via line 142 (which contains a transalkylation reaction product mixture effluent) to a stabilizer 143, where light gas is collected and removed via line 144. A side stream from the stabilizer 143 is recycled to the depentanizer 102 via line 145 (which contains C6A, C7A, and co-boilers) and the stabilizer bottoms are fed to the benzene column 112 via line 146 (which contains C6+ liquid from stabilizer 143), optionally via olefin saturation zone 110.

This disclosure is further illustrated by the following non-limiting examples.

### Examples

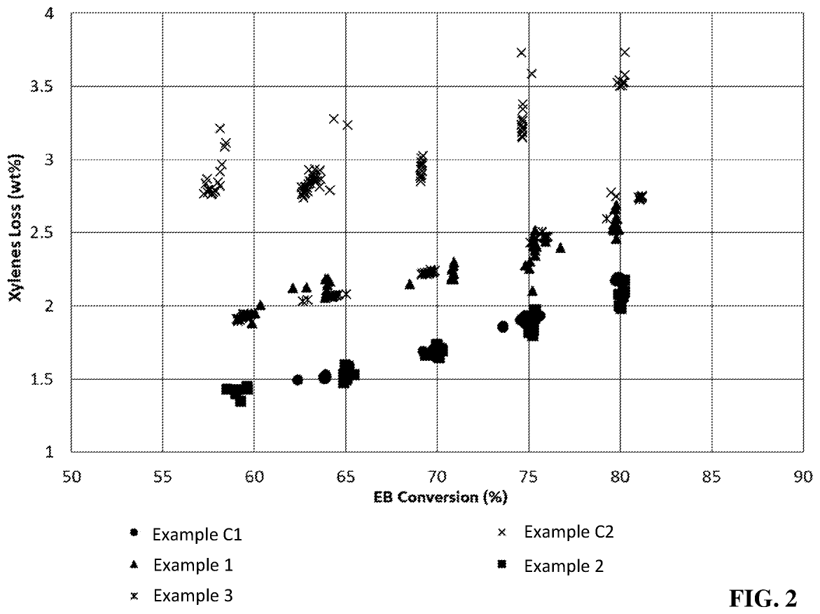

In the following examples, a quantity of a C8 aromatics mixture was fed into a VPI unit, with or without co-feeding an additional quantity of toluene. The C8 aromatics mixture comprised <1 wt % of toluene, based on the total weight of the C8 aromatics mixture. The C8 aromatics mixture comprised p-xylene and EB at various concentrations. The feed mixture contacted with a vapor phase isomerization catalyst system in the VPI unit, underwent isomerization reactions, EB conversion reactions and other reactions under vapor-phase isomerization conditions to produce an isomerization product mixture effluent. All processes in the examples were operated under the same pressure (232 psig, 1600 kPa, gauge), hydrogen to hydrocarbon molar ratio (1:1), and WHSV (15 hour⁻¹). Temperature was adjusted to obtain the desired EB conversion. The quantity of xylenes in the product mixture effluent was determined. Xylenes loss in the process was calculated as the total xylenes concentration in the total feed minus the total xylenes concentration in the product mixture effluent. The compositions of the total feed including the C8 aromatics mixture and any co-fed toluene, total xylenes in the product mixture effluent, and xylenes loss are reported in TABLE 1 below. Additional data are presented in FIGS. 2 to 4.

TABLE 1

| | | | | | Total | |
|---|---|---|---|---|---|---|
| | Total Feed Composition (wt %) | | | | Xylenes in | Xylenes |
| Example | | | | Total | Product | loss |
| No. | Toluene | p-Xylene | EB | Xylenes | (wt %)* | (wt %) |
| C1 | 0.92 | 2.60 | 13.01 | 84.8 | 83.02 | 1.8 |
| C2 | 0.81 | 7.76 | 7.97 | 90.3 | 87.38 | 3.0 |
| 1 | 6.03 | 7.38 | 7.61 | 85.6 | 83.6 | 2.0 |
| 2 | 10.90 | 6.98 | 7.19 | 81.2 | 79.57 | 1.6 |
| 3 | 3.29 | 7.63 | 7.85 | 88.5 | 85.96 | 2.5 |

*At an EB conversion of 75%.

Examples 1 and 2 are comparative examples because the total toluene fed into the VPI unit, including any toluene present in the C8 aromatics mixture, was well below 2 wt %.

From Examples C1 and C2, one can clearly observe that as the p-xylene concentration in the total composition increased from 2.60 wt % in Example to C1 to 2.76 wt % in Example C2, xylenes loss increased from 1.8 wt % to 3.0 wt %, a jump of 67%, while the toluene fed into the VPI unit remained similar and significantly lower than 2 wt %.

Examples 1, 2, and 3 are examples of process of this disclosure in that significant quantity of toluene was co-fed into the VPI unit along with the C8 aromatics mixture, and the total toluene concentrations in the total feed to the VPI unit, including the co-fed additional toluene and any toluene present in the C8 aromatics mixture, was all above 3 wt % in all three examples. The p-xylene concentrations in the total feeds to the VPI unit in Examples C2, 1, 2, and 3 are comparable, and so are the EB concentrations.

As can be clearly seen from TABLE 1, from Example C2, to Example 3, to Example 1, and to Example 2, the total toluene concentration in the total feed to the VPI unit increased from 0.81 wt % in Example C2, to 3.29 wt % in Example 3, to 6.03 wt % in Example 1, and to 10.90 wt % in Example 2. Accordingly, the p-xylenes loss at 75% EB conversion in these four examples decreased from 3.0 wt % in Example C2, to 2.5 wt % in Example 3, to 2.0 wt % in Example 1, and to 1.6 wt % in Example 2. Clearly, as the quantity of toluene fed into the VPI unit increased, the xylenes loss was reduced.

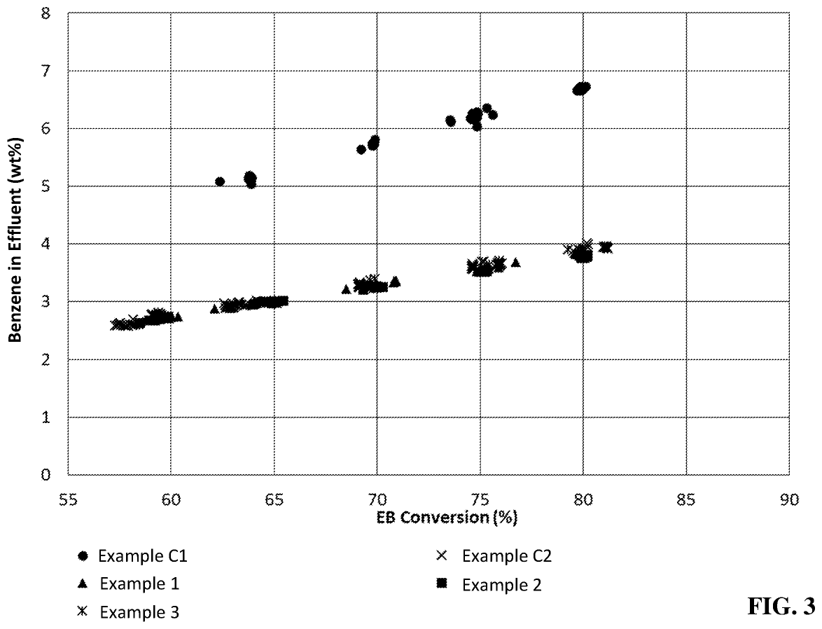

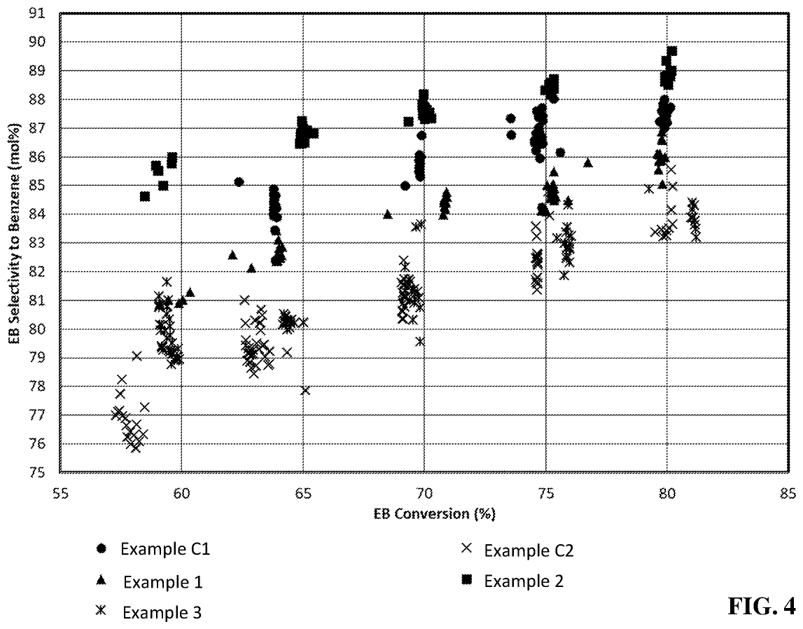

FIG. 2 is a graph showing xylenes loss (wt %) as a function of EB conversion (%) in Examples C1, C2, 1, 2, and 3. FIG. 3 is a graph showing benzene concentration in the isomerization product mixture effluent (wt %) as a function of EB conversion (%) in Examples C1, C2, 1, 2, and 3. FIG. 4 is a graph showing selectivity to benzene (mol %) in the EB conversion process as a function of the EB conversion (%) in Examples C1, C2, 1, 2, and 3.

Examples C2, 1, 2, and 3 have comparable EB concentrations in the total feed supplied in to the VPI unit. As can be seen from FIG. 4, in general, as the toluene concentration in the total feed increased from Example C2, to Example 3, to Example 1, and to Example 2, at EB conversion from 55% to 85%, the selectivity to benzene in EB conversion increased. At the highest toluene co-feed of 10 wt %, Example 2 exhibited a much higher selectivity to benzene in EB conversion than any other examples. Such high selectivity to benzene in EB conversion resulting from a high toluene co-feed quantity is totally unexpected and highly advantageous. The benzene produced can be separated as an aromatic hydrocarbon product, or converted to other valuable products including toluene, xylenes, and the like.

Overall, processes and apparatus of this disclosure can provide production of p-xylene with reduced xylenes loss, increasing EB selectivity to benzene, and increasing benzene production, by featuring an LPI unit and a VPI unit, and one or more toluene co-feed source(s) coupled with the apparatus.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of this disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited.

Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures to the extent they are not inconsistent with this text. As is apparent from the foregoing general description and the specific embodiments, while forms of this disclosure have been illustrated and described, various modifications can be made without departing from the spirit and scope of this disclosure. Accordingly, it is not intended that this disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While this disclosure has been described with respect to a number of embodiments and examples, those skilled in the art, having benefit of this disclosure, will appreciate that other embodiments can be devised which do not depart from the scope and spirit of this disclosure.

What is claimed is:

1. A process for producing p-xylene, the process comprising:
(I) supplying a first C8 aromatics mixture and toluene into a vapor-phase isomerization (“VPI”) unit, wherein:
the first C8 aromatics mixture comprises ethylbenzene, p-xylene, and optionally toluene at a toluene concentration lower than 2 wt %, based on the total weight of the first C8 aromatics mixture; and
the total weight of toluene fed into the VPI unit, including any toluene present in the first C8 aromatics mixture, is at least 2 wt % of the total weight of the C8 aromatics mixture and the toluene fed into the VPI unit;
(II) operating the VPI unit under VPI conditions effective to isomerize the xylenes and dealkylate ethylbenzene in the presence of a first catalyst system in the VPI unit; and
(III) obtaining a first product mixture effluent from the VPI unit, the first product mixture effluent being depleted in ethylbenzene relative to the first C8 aromatics mixture.

2. The process of claim 1, wherein:
the first C8 aromatics mixture comprises p-xylene at a concentration of at least 3 wt %, based on the total weight of the xylenes in the first C8 aromatics mixture.

3. The process of claim 2, wherein:
the first C8 aromatics mixture comprises p-xylene at a concentration of at least 5 wt %, based on the total weight of the xylenes in the first C8 aromatics mixture.

4. The process of claim 1, wherein:
the ratio of the moles of toluene fed into the VPI unit, including any toluene contained in the C8 aromatics mixture, to the moles of p-xylene contained in the C8 aromatics mixture fed into the VPI unit in step (I) is R1, and R1≥0.40.

5. The process of claim 4, wherein R1≥0.50.

6. The process of claim 4, wherein R1≤2.5.

7. The process of claim 1, wherein the weight of toluene fed into the VPI unit, including any toluene contained in the first C8 aromatics mixture, is at least 3 wt % of the total weight of the C8 aromatics mixture and toluene fed into the VPI unit.

8. The process of claim 7, wherein the weight of toluene fed into the VPI unit, including any toluene contained in the first C8 aromatics mixture, is at least 5 wt % of the total weight of the C8 aromatics mixture and the toluene fed into the VPI unit.

9. The process of claim 7, wherein the weight of toluene fed into the VPI unit, including any toluene contained in the first C8 aromatics mixture, is at most 20 wt % of the total weight of the C8 aromatics mixture and the toluene fed into the VPI unit.

10. The process of claim 1, wherein:
(a) the process exhibits a first xylenes loss of Lx(1), where Lx(1)=100%*(W1−W2)/W1, W1 is the aggregate weight of all xylenes present in the first C8 aromatics mixture, W2 is the aggregate weight of all xylenes present in the first product mixture effluent;
(b) a comparative process exhibits a second xylenes loss of Lx(2), wherein the comparative process is identical to the process in (a) except the toluene is not supplied in (I), and in the comparative process, the first C8 aromatics mixture comprises 0.9 wt % of toluene based on the total weight of the first C8 aromatics mixture, Lx(2)=100%*(W3−W4)/W3, W3 is the aggregate weight of all xylenes present in the first C8 aromatics mixture, and W4 is the aggregate weight of all xylenes present in the first product mixture effluent produced from the comparative process; and
(c) 0.2%≤Lx(2)−Lx(1)≤1.5%.

11. The process of claim 1, further comprising:
(VII) supplying a second C8 aromatics mixture into a liquid-phase isomerization unit (“LPI”), the second C8 aromatics mixture comprising m-xylene and/or o-xylene;
(VIII) operating the LPI unit under conditions effective to isomerize m-xylene and/or o-xylene in the presence of a second catalyst system in the LPI unit; and
(IX) obtaining a second product mixture effluent from the LPI unit, the second product mixture effluent being p-xylene rich relative to the second C8 aromatics mixture.

12. The process of claim 11, wherein the first C8 aromatics mixture and the second C8 aromatics mixture have substantially the same composition.

13. The process of claim 11, wherein the weight of the first C8 aromatics mixture fed to the VPI unit in step (I) is Q1, the weight of the second C8 aromatics mixture fed to the LPI unit in step (VII) is Q2, the ratio of Q2/Q1 is R3, and R3≥0.25.

14. The process of claim 13, wherein R3≥0.5.

15. The process of claim 1, wherein the EB conversion in step (II) is ≥50%, preferably ≥60%, more preferably ≥70%.

16. The process of claim 1, wherein the EB conversion in step (II) exhibits a benzene selectivity ≥70%, preferably ≥75%, more preferably ≥80%, and still more preferably ≥85%.

17. A process for producing p-xylene, the process comprising:
(A) supplying a first C8 aromatics mixture into a vapor-phase isomerization (“VPI”) unit having a first catalyst system disposed therein, the first C8 aromatics mixture comprising ethylbenzene, p-xylene, and optionally toluene at a toluene concentration lower than 2 wt % based on the total weight of the first C8 aromatics mixture;
(B) operating the VPI unit under conditions effective to isomerize at least a portion of the xylenes and dealkylate at least a portion of the ethylbenzene in the presence of the first catalyst system;
(C) obtaining a first product mixture effluent from the VPI unit, the first product mixture effluent being depleted in ethylbenzene relative to the first C8 aromatics mixture;
(D) determining a concentration of p-xylene of C(pX) in the first C8 aromatics mixture based on the total weight of C8 aromatics mixture; and
(E) if C(pX)≥5 wt %, supplying additional toluene to the VPI unit such that the total weight of toluene fed into the VPI unit, including any toluene contained in the first aromatics mixture, is at least 2 wt % of the total weight of the C8 aromatics mixture and the toluene fed into the VPI unit.

18. The process of claim 17, wherein:
the ratio of the moles of toluene fed into the VPI unit to the moles of the p-xylene contained in the first C8 aromatics mixture fed into the VPI unit in step (A) is R1, and R1≥0.40, preferably R1≥0.50.

19. The process of claim 18, wherein R1≤2.50.

20. The process of claim 16, wherein the weight of toluene fed into the VPI unit, including any toluene present in the first aromatics mixture, is in a range from 3 to 20 wt %, preferably from 5 to 15 wt %, of the total weight of the C8 aromatics mixture and the toluene fed into the VPI unit.

21. The process of claim 16, wherein:
(a) the process exhibits a first xylenes loss of Lx(1) when additional toluene is supplied in (E), where Lx(1)=100%*(W1−W2)/W1, W1 is the aggregate weight of all xylenes present in the first C8 aromatics mixture, W2 is the aggregate weight of all xylenes present in the first product mixture effluent;
(b) a comparative process exhibits a second xylenes loss of Lx(2), wherein the comparative process is identical to the process in (a) except no additional toluene is supplied in (E), and in the comparative process the first C8 aromatics mixture comprises 0.9 wt % of toluene based on the total weight of the first C8 aromatics mixture, Lx(2)=100%*(W3−W4)/W3, W3 is the aggregate weight of all xylenes present in the first C8 aromatics mixture, and W4 is the aggregate weight of all xylenes present in the first product mixture effluent produced from the comparative process; and
(c) 0.2%≤Lx(2)−Lx(1)≤1.5%.

22. The process of claim 16, further comprising:
(VII) supplying a second C8 aromatics mixture into a liquid-phase isomerization (“LPI”) unit equipped with a second catalyst system, the second C8 aromatics mixture comprising m-xylene and/or o-xylene;
(VIII) operating the LPI unit under conditions effective to isomerize m-xylene and/or o-xylene in the presence of the second catalyst system; and
(IX) obtaining a second product mixture effluent from the LPI unit, the second product mixture effluent being p-xylene rich relative to the second C8 aromatics mixture.

23. The process of claim 22, wherein the first C8 aromatics mixture and the second C8 aromatics mixture have substantially the same composition.

24. The process of claim 22, wherein the weight of the first C8 aromatics mixture fed to the VPI unit in step (I) is Q1, the weight of the second C8 aromatics mixture fed to the LPI unit in step (II) is Q2, the ratio of Q2/Q1 is R3, and R3≥0.25.

25. The process of claim 24, wherein R3≥0.5.
